(19) United States
(12) Patent Application Publication
(10) Pub. No.: US 2022/0386917 A1
(43) Pub. Date: Dec. 8, 2022

(54) Urinary Output Measuring System

(71) Applicant: C. R. Bard, Inc., Franklin Lakes, NJ (US)

(72) Inventors: Gregory Mann, Mcdonough, GA (US); Damien Marechal, Claix (FR); Yolanda Rhodes, Cypress, TX (US)

(21) Appl. No.: 17/833,682

(22) Filed: Jun. 6, 2022

Related U.S. Application Data
(60) Provisional application No. 63/208,438, filed on Jun. 8, 2021.

(51) Int. Cl. A61B 5/20; A61B 10/00; A61B 5/00

(52) U.S. Cl. CPC A61B 5/208; A61B 10/007; A61B 5/6891; A61B 5/7246; A61B 2560/0223; G08B 21/182

(57) ABSTRACT

Disclosed herein is a urine output measuring system including a urine hat having a urine hat body including a rim, the urine hat defining a cavity configured to receive a volume of urine, a plurality of feet defining a platform configured to be coupled to a toilet to suspend the urine hat, and one or more sensors coupled to the platform, the one or more sensors being in communication with a console and configured to detect and transmit one or more load values to the console.

### PRIORITY

This application claims the benefit of priority to U.S. Provisional Application No. 63/208,438, filed Jun. 8, 2021, which is incorporated by reference in its entirety into this application.

### BACKGROUND

Measuring urinary output, measuring urinary output flow rate, and collecting urine samples can be difficult for ambulatory patients. Some systems require patients to void urine into a urine hat, transfer the urine hat to a laboratory for analysis or leave the urine hat in place for a clinician to measure and analyze. Without clinician oversite, patients may be prone to disrupting the urine collection process, thereby prevent accurate measurement or analysis. It would be beneficial to patients and clinicians to have a system that measures and analyzes urine output during or immediately after collection and that has safeguards to ensure the entire volume of urine is collected and measured. Disclosed herein are systems and methods that address the foregoing.

### SUMMARY

Disclosed herein is a urinary output measuring system that, according to some embodiments, includes: a urine hat having a body defining a cavity configured to collect urine therein, the body including a rim extending radially outward from the body; a platform including a plurality of feet coupled together via connecting members, the feet configured for coupling the platform to a toilet bowl, the platform coupled with the urine hat so as to suspend the urine hat over the toilet bowl; one or more sensors coupled to the platform, the one or more sensors configured to determine a load value exerted on the platform by the urine hat. The system further includes a console including one or more processors and a non-transitory computer-readable storage medium having logic stored thereon that, when executed by the one or more processors, performs operations that include receiving the load value from the one or more sensors; and determining a volume of the collected urine from the load value.

In some embodiments, the plurality of feet includes the one or more sensors.

In some embodiments, the plurality of feet are configured to secure the platform to the toilet bowl via a snap fit, or an interference fit.

In some embodiments, the rim is configured to operatively couple the urine hat to the platform.

In some embodiments, the system further includes a monitor coupled to the platform, where the monitor is in communication with the console, and where the monitor is further configured to display information pertaining to the collection of urine.

In some embodiments, the console is configured to wirelessly communicate with an external computing device.

In some embodiments, the operations further include correlating the determined volume of the collected urine with a time of day and transmitting the correlated volume of collected urine to one or more of the monitor, or the external computing device.

In some embodiments, the operations further include transmitting the correlated volume of collected urine to the external computing device, where the external computing device is coupled with an electronic medical record system.

In some embodiments, the operations further include calibrating the one or more sensors.

In some embodiments, the operations further include generating an alert pertaining to one or more operating conditions of the system and transmitting the alert to at least one of the monitor or the external computing device.

In some embodiments, the operating conditions include one or more of: a coupling status of the urine hat with the platform; a disruption in the collection of urine; the volume of collected urine exceeding a predefined volume; or a status of sensor calibration. In some embodiments, the operating conditions include one or more operating conditions defined by a user of the system.

In some embodiments, the operations further include: (i) receiving a first load value from the one or more sensors; (ii) correlating the first load value with a first time of day; (iii) receiving a second load value from the one or more sensors; (iv) correlating the second load value with a second time of day different from the first time of day; and (v) determining a volumetric rate of the collected urine from the first load value correlated with the first time of day and the second load value correlated with the second time of day.

In some embodiments, the urine hat includes a drain port located at a bottom of the cavity, the drain port selectively transitionable between: (i) a closed position preventing collected urine from exiting the urine hat through the drain port, and (ii) an open position allowing collected urine to exit the urine hat through the drain port.

Also disclosed herein is method of measuring urine output of a patient, that according to some embodiments, includes: (i) collecting urine output from the patient within a urine hat of a urine output measuring system; (ii) determining a load value exerted on a platform of the urine output measuring system; and (iii) determining a volume of the collected urine output from the load value.

In some embodiments, the method further includes correlating the volume of collected urine output with a time of day.

In some embodiments, the method further includes transmitting the correlated volume of collected urine output to one or more of a monitor of the urine output measuring system or an external computing device.

In some embodiments, the method further includes transmitting the correlated volume of collected urine output to an electronic medical record system.

In some embodiments, the method further includes calibrating one or more sensors of the urine output measuring system.

In some embodiments, the method further includes generating an alert pertaining to operation of the urine output measuring system and transmitting the alert to a monitor of the urine output measuring system and/or an external computing device.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a urinary output measuring system, in accordance with some embodiments;

FIG. 2 illustrates a block diagram of a console and related components of the system of FIG. 1, in accordance with some embodiments;

FIG. 3 illustrates a cross-sectional side view of the urine hat of FIG. 1, in accordance with some embodiments;

FIG. 4A illustrates a top view of the system of FIG. 1 with the urine hat decoupled from the platform, in accordance with some embodiments;

FIG. 4B illustrates a top view of the system of FIG. 1 with the urine hat coupled with the platform, in accordance with some embodiments;

FIGS. 5A-5B illustrate a cross-sectional side view of the system of FIG. 1 depicting an exemplary method of measuring a volume of urine output collected within the urine hat, in accordance with some embodiments; and

FIG. 6 illustrates a flow chart of an exemplary method of measuring a urine output collected within the urine hat, in accordance with some embodiments

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

The phrases “connected to,” “coupled with,” and “in communication with” refer to any form of interaction between two or more entities, including but not limited to mechanical, electrical, or urine interaction. Two components may be coupled with each other even though they are not in direct contact with each other. For example, two components may be coupled with each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

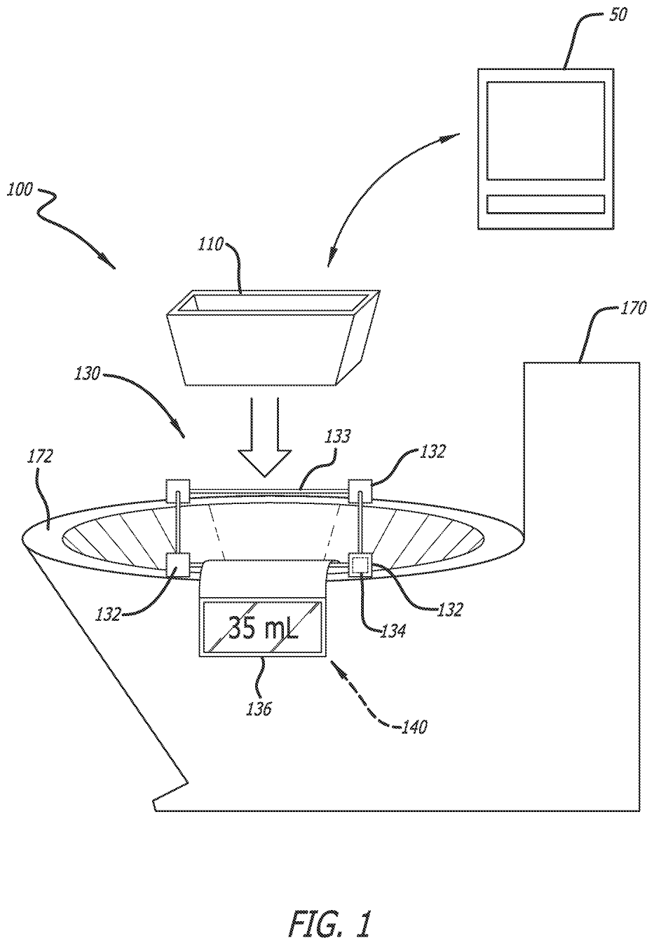

FIG. 1 illustrates a perspective view of the urinary output measuring system 100, in accordance with some embodiments. The urinary output measuring system (“system”) 100 is generally configured for use with a toilet 170 so that a urine output (i.e., a volume of excreted urine) of a user/patient may be captured/collected, measured, and/or analyzed. In some embodiments, the system 100 may be configured to collect and measure a bodily fluid or substance other than urine. The system 100 generally includes a urine hat 110 and a platform 130, where the urine hat 110 is selectively coupleable with the platform 130. The urine hat 110 defines an open container configured to collect therein a urine output from the user. The platform 130 is configured to be secured to a toilet bowl 172 of a toilet 170. When coupled with the platform 130, the urine hat 110 is suspended from the platform 130 so as to extend across the toilet bowl 172. The platform 130 is configured to determine a volume of the urine output collected within the urine hat 110. In some embodiments, the determined volume (e.g., amount) of the urine output may be transmitted to an external computing device 50. The external computing device 50 may be any type of an external device include a network, a personal computer, a tablet, or a cell phone. The external computing device 50 include or be component of an electronic medical record (“EMR”) system.

The urine hat 110 is configured to engage (e.g., contact) the platform 130 in two or more locations. The platform 130 may be configured to couple with the toilet bowl 172 or a seat of the toilet 170. In the illustrated embodiment, the platform 130 includes a plurality (e.g., 3, 4 or more) of feet 132 configured to contact the toilet bowl 172 (e.g., a rim of the toilet bowl 172) to stabilize the platform 130 during use. In some embodiments, the feet 132 (or platform 130 generally) may be configured for securement to the toilet bowl 172 via an interference fit, a snap fit, a press fit, an adhesive, or the like. Other methods of securing the platform 130 to the toilet bowl 172 are also considered, such as straps, for example. In some embodiments, the feet 132 may be arranged in a square, a rectangle, or the like across the opening of the toilet bowl 172, as illustrated in FIG. 1.

In the illustrated embodiment, the feet 132 may be physically coupled to each other via connecting members 133 to define a structure or frame of the platform 130 as illustrated in FIG. 1. The connecting members 133 may include flexible members such as cables or wires, for example, or the connecting members 133 may be rigid members such as plastic beams, for example. In some embodiments, a length of the connecting members 133 may be adjustable to accommodate different sizes and shapes of the toilet bowl 172. In other embodiments, the connecting members 133 may be omitted, i.e., the platform 130 may comprise the plurality of feet 132 individually attached to the toilet bowl 172, where the plurality of feet 132 are physically coupled with each other only indirectly via the toilet bowl 172.

The platform 130 includes a number (e.g., 1, 2, 3, 4 or more) of sensors 134 configured to obtain various measurements of the urine collected within the urine hat 110 when the urine hat 110 is suspended from the platform 130 as further described below. The sensors 134 may be included with one or more of the feet 132, or the connecting members 133. In some embodiments, the sensors 134 may include one or more load sensors including strain sensors, strain gauges, piezoelectric load cells, or the like. In the illustrated embodiment, each sensor 134 is a load sensor, although other types of sensors are considered. In the illustrated embodiment, all or a subset of the feet 132 may include a sensor 134. In other embodiments, the load sensor 134 may be incorporated into all or a subset of the connecting members 133. By way of summary, the platform 130 includes load sensors 134 configured to determine a load (e.g., downward force) exerted onto the platform 130 by the urine hat 110 during use, where the load is at least partially defined by a weight of urine output contained within the urine hat 110.

In the illustrated embodiment, the system 100 includes a monitor 136 configured to display information related to operation of the system 100. In some embodiments, the monitor 136 may be physically attached to the platform 130 such that the monitor 136 is supported by the platform 130. For example, the monitor 136 may hang from the platform 130 so as to be visible on a front or a side of the toilet 170. In other embodiments, the monitor 136 may be physically coupled to the platform 130 via an electrical cable/cord so that the monitor 136 may be spaced away from the platform 130, such as placed on a shelf, or hung on a wall, for example. In still other embodiments, the monitor 136 may be physically separated from (i.e., not physically attached to) the platform 130. The monitor 136 is communicatively coupled with the sensors 134 so that the monitor 136 may receive load data from the sensors 134. The monitor 136 may be coupled with the sensors 134 via a wired or wireless connection. For example, the sensors 134 may be “smart” sensors having a capability to wirelessly transmit load data to the monitor 136.

The system 100 may include a console 140 in communication with the sensors 134 and the monitor 136. The console 140 may be wired to the one or more sensors 134 and/or the monitor 136 or may be in wireless communication with the one or more sensors 134 and/or the monitor 136. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. In some embodiments, the console 140 may be physically coupled with or incorporated within the monitor 136. In other embodiments, the console 140 may be physically coupled with or incorporated into the platform 130, such as the plurality of feet 132 or the connecting members 133, for example. In still other embodiments, the console 140 may be a separate component physically coupled with the toilet 170. The console 140 may be configured to (i) receive load data (e.g., electrical signals) from the sensors 134, (ii) process the load data, and (ii) transmit information pertaining to the load data to the monitor 136 to be displayed by the monitor 136. In some embodiments, the console 140 may be configured to record (e.g., store in memory) the information pertaining to the load data. In some embodiments, the console 140 may be configured to wirelessly transmit the load data or other information/data to the external computing device 50, including the EMR system.

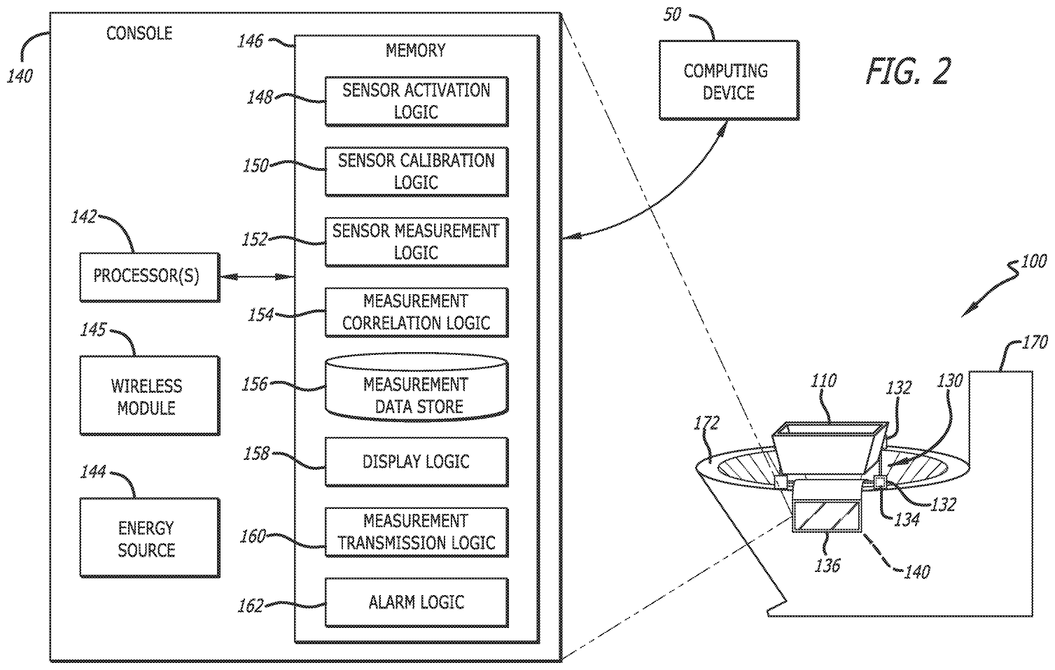

FIG. 2 illustrates a block diagram of the console 140 and other components of the system 100, in accordance with some embodiments. In some embodiments, the console 140 may include one or more processors 142, an energy source 144, a wireless module 145, a non-transitory computer-readable storage medium (“memory”) 146. In some embodiments, the energy source 144 may include a rechargeable battery and/or an external facility power source. The energy source 144 may provide power to the sensors 134 and/or the monitor 136. The wireless module 145 may be configured to wirelessly exchange data with one or more of the monitor 136, the sensors 134, or the external computing device 50.

The memory 146 includes a plurality of logic modules that may include a sensor activation logic 148, a sensor calibration logic 150, a sensor measurement logic 152, a measurement correlation logic 154, a measurement data store 156, a display logic 158, a measurement transmission logic 160, and an alarm logic 162. In some embodiments, the sensor activation logic 148 may be configured to activate the sensors 134, such as provide power to the sensors 134 and/or initiate communication with the sensors 134, for example. In some embodiments, the one or more sensors 134 may be activated in accordance with a timer of the sensor activation logic 148. In an embodiment, the sensor activation logic 148 may activate the sensors 134 in response to a user input, such as via an actuator (e.g., a button or the like) operatively coupled with the console 140, and which may be physically coupled to the monitor 136. In some embodiments, the one or more sensors 134 may be activated when the urine hat 110 is placed upon the platform 130. For example, the sensor activation logic 148 may place the sensors 134 in a standby mode when the urine hat 110 decoupled from the platform 130. In the standby mode, the sensor activation logic 148 may detect coupling of the urine hat 110 to the platform 130, whereupon sensor activation logic 148 may transition the sensors 134 from the standby mode to an activated mode.

In some embodiments, the sensor calibration logic 150 may be configured to calibrate (e.g., tare) the one or more sensors 134, e.g., run a calibration routine. In some embodiments, the calibration logic 150 may calibrate the one or more sensors 134 upon activation of the one or more sensors 134. In other embodiments, the calibration logic 150 may calibrate the one or more sensors 134 in response to a user input. In some embodiments, the sensor calibration logic 150 may calibrate the one or more sensors 134 upon detection of a tare load applied to the one or more sensors 134, where in some embodiments, the tare load is a load defined by an empty urine hat 110.

The sensor measurement logic 152 may be configured to receive/extract load values/data from the sensors 134. In some embodiments, the sensor measurement logic 152 may continuously extract the load data. In some embodiments, the sensor measurement logic 152 extract load data in accordance with a defined time interval (e.g., every 3 seconds, every 5 seconds, or the like). It can be appreciated that greater or lesser time interval increments are also contemplated.

The measurement correlation logic 154 may be configured to determine a volume of urine output collected within the urine hat 110 based on the load data. For example, the measurement correlation logic 154 may determine (e.g., calculate) the volume of urine output collected within the urine hat 110 based on the load data in accordance with a defined density of the urine since the load data is at least partially related to a weight of the urine output collected within the urine hat 110. During the urine collection process, the measurement correlation logic 154 may determine the volume of collected urine output multiple times in accordance with the extraction of load data so as to generate multiple determined volumes of urine output.

The measurement correlation logic 154 may also correlate the determined volume of urine output with a time of day. In other words, the measurement correlation logic 154 may attach a time stamp to each determined volume of urine output. As such, the measurement correlation logic 154 may be configured to calculate a rate of urine output flowing into the urine hat 110. For example, the measurement correlation logic 154 may determine a first volume of urine output and correlate the first determined volume with a first time of day. The measurement correlation logic 154 may then determine a second volume of urine output and correlate the second determined volume with a second time of day. The measurement correlation logic 154 may then determine a rate of urine output based the first determined volume of urine output correlated with the first time of day and the second determined volume of urine output correlated with the second time of day. In some embodiments, the measurement correlation logic 154 may be configured to calculate or perform additional measurements or analysis from the determined volumes, such as the total urine output for day, or a maximum or minimum daily urine output over multiple days, for example.

In some embodiments, the measurement correlation logic 154 may monitor the load values received from the sensors 134 to determine when a complete urine output has been collected. For example, when the measurement correlation logic 154 receives multiple identical load values over a time interval or multiple load values within a threshold range over a time interval, the console 140 may determine that the patient is finished excreting urine into the urine hat 110.

In some embodiments, the measurement data store 156 may be configured to store information and/or data related to operation of the system 100. Exemplary information and/or data may include raw load data from the sensors 134, determined urine output volumes, determined urine output volumes correlated with the time of day, determined urine output rate values, and alerts, for example.

In some embodiments, the display logic 158 may be configured to display urine collection information pertinent to the user, such as a current determined urine output volume collected in the urine hat 110, a running total of urine output for a given day, a rate of the urine output over a defined time period, and alerts, for example. The display logic 158 may also display any of the information and/or data stored within the measurement data store 156. In some embodiments, the display logic 158 may be configured to display portions of the urine collection information continuously (i.e., during collection of urine output), such as the determined urine output volume collected in the urine hat 110, for example. The display logic 158 may also be configured to display other information at the conclusion of event, such as after a urine output session, or at the end of a day, for example.

In some embodiments, the measurement transmission logic 160 may be configured to wireless transmit the urine collection information to the computing device and/or the ERM system using wireless communication modalities as describe above.

In some embodiments, the alarm logic 162 may be configured to generate an audible alert or alarm, such as in the case of a user defined parameter that is either achieved or not achieved. For example, the alarm logic 162 may be configured to generate an alarm to notify the user or a clinician if the urine hat 110 is disturbed during urine collection, such as the user disposing of urine before the full urine output is collected, for example. The alarm logic 162 may be configured to generate an alarm if the plurality of feet 132 are disturbed during urine capture. The user may configure an alarm if the detected load values are decreasing during urine capture, indicating urine is leaving the urine hat 110. The alarm logic 162 may be configured to generate an alarm if the one or more sensors 134 are not properly calibrated.

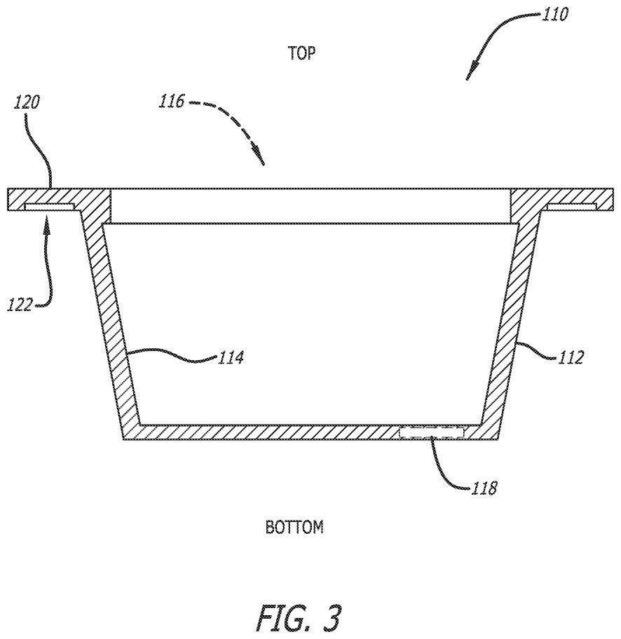

FIG. 3 illustrates a cross-sectional view of the urine hat 110, in accordance with some embodiments. The urine hat 110 includes a urine hat body 112 defining a cavity 114, configured to receive urine output therein through a top opening 116. In some embodiments, the urine hat 110 may have the shape of a rectangular prism, an upside down trapezoidal prism, a trough, a hemisphere or the like.

In some embodiments, the urine hat 110 may include a rim 120 extending radially outward along a top side of the urine hat 110. In some embodiments, the rim 120 may extend along an entire circumference of the urine hat 110. In other embodiments, the rim 120 may include separate rim sections, such a two rim sections extending away from the body 114 in opposite directions. The rim 120 may be configured to rest on, or otherwise engage, the platform 130. In some embodiments, the rim 120 may directly engage two or more of the connecting members 133. In some embodiments, the rim 120 may directly engage one or more of the feet 132. In some embodiments, the rim 120 may include a ridge or a groove 122 on the bottom side of the rim 120. In some embodiments, the ridge 122 may be configured to stabilize a lateral position of the urine hat 110 with respect to the platform 130 while maintaining a detachability of the urine hat 110 from the platform 130. In some embodiments, the ridge 122 may enable coupling of the urine hat 110 with the platform 130 via a press fit, a snap fit, an interference fit, or the like. In some embodiments, the urine hat 110 may be disposable or reusable. In some embodiments, the urine hat 110 may be formed contiguously or formed from multiple parts coupled together. In some embodiments, the urine hat 110 may be 3D printed, injection molded, thermoformed, or the like. In some embodiments, the urine hat 110 may be formed of a thermoplastic material. During use, the user may decouple the urine hat 110 from the platform 130 for any number of reasons, such as pouring the collected urine into the toilet, transporting the urine to another location for sampling, cleaning the urine hat 110, or replacing the urine hat 110, for example.

In some embodiments, the urine hat 110 may optionally include a drain port 118 located at the bottom of the cavity 114. The drain port 118 may be selectively transitionable between an open state and a closed state. For example, the drain port 118 may disposed in the closed state as a default state to ensure that all urine output is collected within the urine hat 110. During use, the drain port 118 may be selectively transitioned to the open state to drain urine from the urine hat 110. In some embodiments, the drain port 118 may be transitionable by the user between the closed state and the open state while the urine hat 110 is coupled with the platform 130.

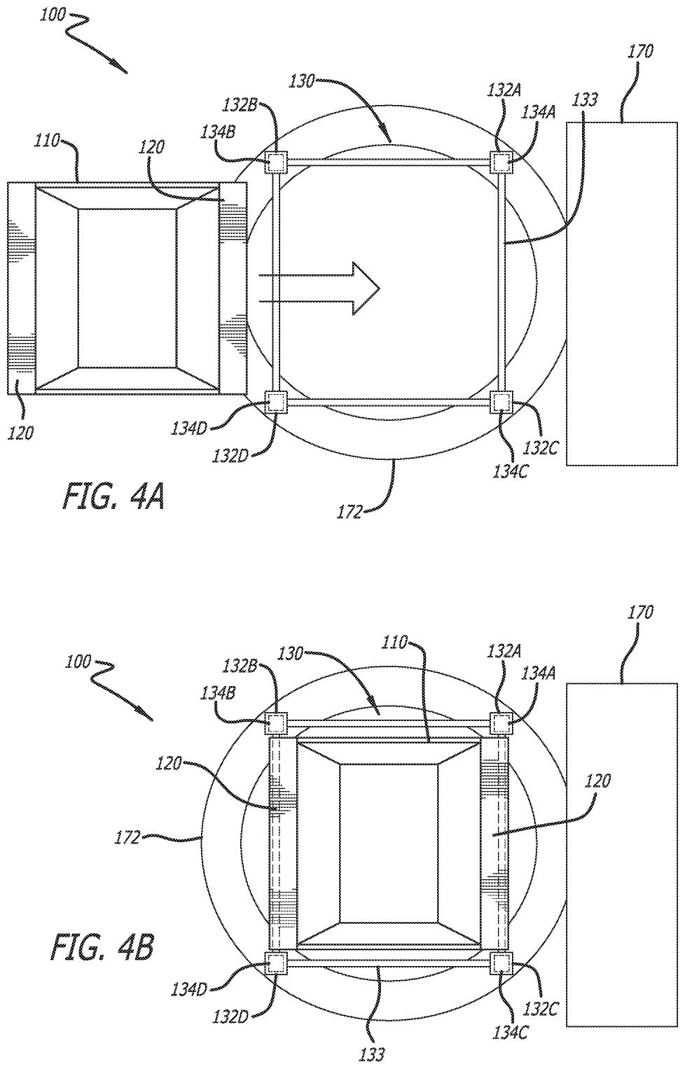

FIGS. 4A-4B illustrate a plan view of the system 100 depicting an exemplary method of coupling a urine hat 110 to the platform 130, in accordance with some embodiments. In some embodiments, the platform 130 may be initially coupled toilet 172 bowl as illustrated in FIG. 4A. In some embodiments, the platform 130 may span across a portion of or the entirety toilet bowl 172, allowing a maximum area of the toilet bowl 172 to capture urine from the patient. Coupling the platform 130 to the toilet bowl 172 may include coupling the plurality of feet 132A-132D, having the one or more sensors 134A-134D, directly coupled to the toilet bowl 172, such as along a rim of the toilet bowl 172. The platform 130 may be configured in a rectangular shape, a square shape or the like. As illustrated in FIG. 4B, the user may place the urine hat 110 on the platform 130, so that the urine hat 110 engages the plurality of feet 132A-132D including the one or more sensors 134A-134D. In some embodiments, the user may place the urine hat 110 on the platform 130, so that the rim 120 of the urine hat 110 engages the plurality of feet 132A-132D. In an embodiment, the user may place the urine hat 110 on the platform 130, so that the rim 120 of the urine hat 110 only engages the connecting members 133 between the plurality of feet 132A-132D. In some embodiments, the user may detachably couple the urine hat 110 to the platform 130 as described above.

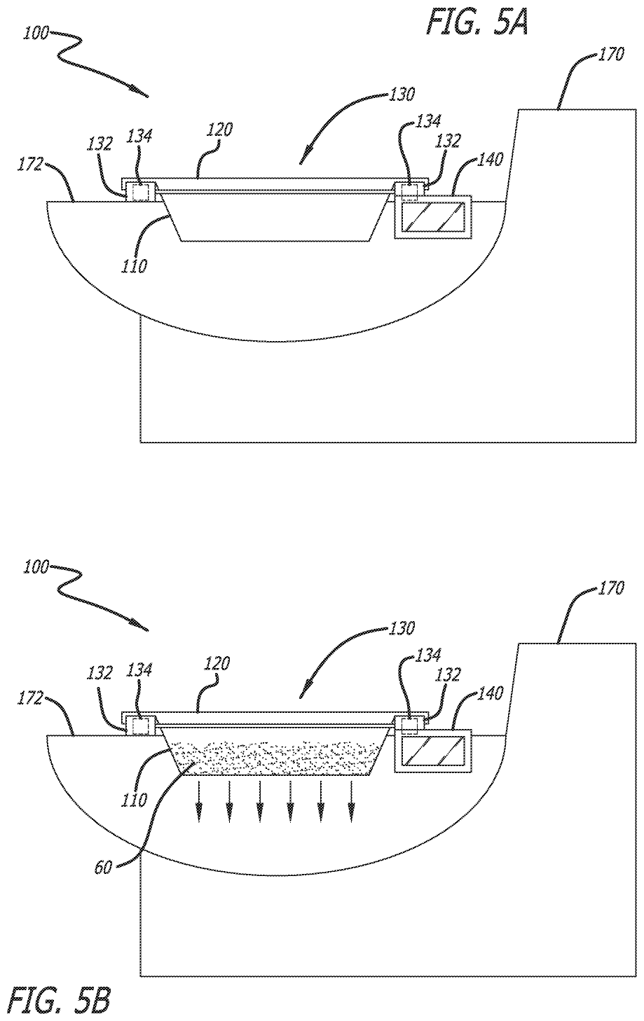

FIGS. 5A-5B illustrate a cross-sectional view of system of FIG. 1 depicting an exemplary method of capturing and measuring urine output within the urine hat 110 using the system 100, in accordance with some embodiments. In some embodiments, the urine hat 110 may be coupled with the platform 130, as illustrated in FIG. 5A, after the platform 130 has been coupled to the toilet bowl 172 as described above. In some embodiments, before collecting any urine output within the urine hat 110, the logic of the system 100 may calibrate the one or more sensors 134.

The user may then excrete urine for collection within the urine hat 110, as illustrated in FIG. 5B. As the volume of urine 60 collected within the urine hat 110 increases, the weight of the urine hat 110 increases, resulting in an increasing the load exerted onto the one or more sensors 134. The one or more sensors 134 may be configured to detect load values and transmit the load values to the console 140. The logic of the console 140 may determine a volume of urine output 60 detected for each detected load value and correlate the volume of urine output 60 a time of day, as described above. The one or more sensors 134 may detect load values and transmit the load values to the console 140 on a time interval or may continuously detect and transmit load values to the console 140. In some embodiments of the method, the logic of the console 140 may monitor the load values received from the sensors 134 to determine when a complete urine output has been collected. For example, the logic of the console 140 may determine that the patient has finished excreting urine when multiple load within a defined threshold range are received over a time interval. The logic of the console 140 may then transmit the one or more of the detected load values, the determined volumes, or corresponding times of day to the computing device or the EMR system.

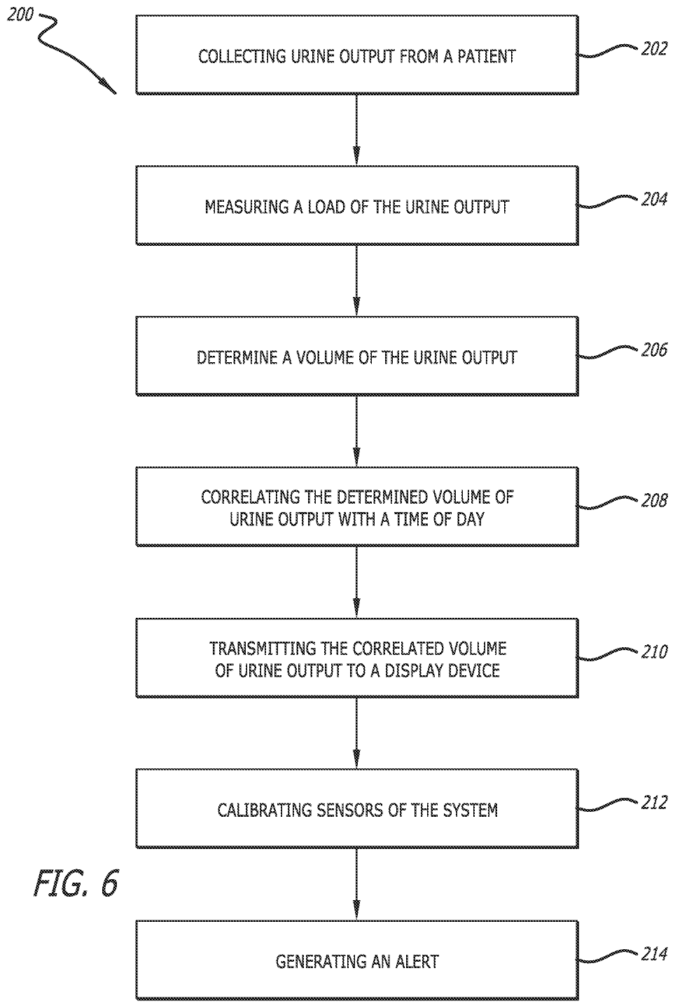

FIG. 6 illustrates a flow chart of an exemplary method 200 of a urine output measuring system 100 measuring a urine output from a patient, in accordance with some embodiments. The method may include all or any subset of a number of steps or actions for performing the method as described below. The steps and/or actions as described below may be interchanged with one another, i.e., unless a specific order of the steps is required for proper operation of the embodiment, the order steps may be modified.

The method includes collecting urine output from the patient within the urine hat 110 of the urine output measuring system 100 (block 202). During use, the user may couple the system with a toilet so that the urine hat 110 is positioned to collect the urine output from the patient. Once positioned, the urine hat 110 may collect the urine output within the cavity 114 of the urine hat 110.

The method further includes determining a load value exerted on a platform of the urine output measuring system 100 (block 204). The urine output collected within the urine hat 110 defines a load (e.g., a weight) of urine within the urine hat 110 and the load exerts a downward force on the platform 130. The sensors 134 of the platform 130 measure/determine the load to define a load value based on the urine output.

The method further includes determining a volume of the collected urine output from the load value (block 206). In some instances, it may be advantageous for a user or a clinician to have the amount of urine output to be defined in terms of volume (e.g., milliliters, liters, or the like.) As such, the urine output measuring system 100 may determine a volume of the urine output from the load value (e.g., the system may calculate a volume of the urine output from the load value utilizing a defined density of the urine).

The method further includes correlating the volume of collected urine output with a time of day (block 208). In some instances, it may be advantageous for the user or the clinician to associate the determined volume of the urine output with a time of day. As such, in some instances, system may assign a time stamp to the determined volume of urine output to define a correlated volume of urine output. Having a time stamp associated with multiple determined volumes of urine output enables the system to calculate a rate of excretion. As such, in some embodiments, the method may include determining the rate of urine excretion.

In some embodiments, the method may further include transmitting the correlated volume of urine output to one or more of a monitor of the urine output measuring system 100 or an external computing device 50 (block 2010). Having determined the volume of urine output, the system 100 may communicate the determined volume of urine output to the user or the clinician. As such, the system 100 may transmit the determined volume of urine output to an output device having a display such as the monitor 136, the external computing device 50 for portraying the correlated volume of urine output, and/or any other information related to the collected urine output.

In some embodiments, the external computing device may be included within an electronic medical record system. As such, the method may further include transmitting the correlated volume of urine output to an electronic medical record system.

In some embodiments, the method further includes calibrating one or more sensors 134 of the system 100 (block 212). In some embodiments, calibrating (e.g., taring) the sensors 134 may enhance an accuracy of the system 100. As such, the system 100 may calibrate the sensors 134 (e.g., run a calibration routine) to ensure measurement accuracy of the urine output.

In some embodiments, the method may further include generating an alert pertaining to operation of the system 100 and transmitting the alert to the monitor 136 and/or the external computing device 50 (block 214). In some instances, the urine output collection process may be interrupted or otherwise disturbed such that an error in the determination of the urine output volume may result. As such, the system 100 may (i) detect the disturbance of the urine output collection process, (ii) generate an alert associated with the disturbance, and (iii) transmit the alert to the monitor 136 and/or the external computing device 50 to provide notification to the user and/or clinician.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A urine output measuring system, comprising:
a urine hat having a body defining a cavity configured to collect urine therein;
a platform including a plurality of feet coupled together via connecting members, the feet configured for coupling the platform to a toilet bowl, the platform coupled with the urine hat so as to suspend the urine hat over the toilet bowl;
one or more sensors coupled to the platform, the one or more sensors configured to determine a load value exerted on the platform by the urine hat; and
a console including one or more processors and a non-transitory computer-readable storage medium having logic stored thereon that, when executed by the one or more processors, performs operations including:
receiving the load value from the one or more sensors; and
determining a volume of the collected urine from the load value.

2. The system according to claim 1, wherein the plurality of feet includes the one or more sensors.

3. The system according to claim 2, wherein the plurality of feet are configured to secure the platform to the toilet bowl via a snap fit, or an interference fit.

4. The system according to claim 1, wherein the urine hat includes a rim extending radially outward from the body, the rim configured to operatively couple the urine hat to the platform.

5. The system according to claim 1, further comprising a monitor coupled to the platform, wherein the monitor:
is in communication with the console, and
is configured to display information pertaining to the collection of urine.

6. The system according to claim 5, wherein the console is configured to wirelessly communicate with an external computing device.

7. The system according to claim 6, wherein the operations further include:
correlating the determined volume of the collected urine with a time of day; and
transmitting the correlated volume of collected urine to one or more of the monitor, or the external computing device.

8. The system according to claim 6, wherein:
the operations further include transmitting the correlated volume of collected urine to the external computing device; and
the external computing device is coupled with an electronic medical record system.

9. The system according to claim 1, the operations further include:
calibrating the one or more sensors.

10. The system according to claim 6, the operations further include:
generating an alert pertaining to one or more operating conditions of the system; and
transmitting the alert to the monitor and/or the external computing device.

11. The system according to claim 10, wherein the operating conditions include one or more of:
a coupling status of the urine hat with the platform;
a disruption in the collection of urine;
the volume of collected urine exceeding a predefined volume; or
a status of sensor calibration.

12. The system according to claim 10, wherein the operating conditions include one or more operating conditions defined by a user of the system.

13. The system according to claim 1, the operations further include:
receiving a first load value from the one or more sensors;
correlating the first load value with a first time of day;
receiving a second load value from the one or more sensors;
correlating the second load value with a second time of day different from the first time of day; and
determining a volumetric rate of the collected urine from the first load value correlated with the first time of day and the second load value correlated with the second time of day.

14. The system according to claim 1, wherein the urine hat includes a drain port located at a bottom of the cavity, the drain port selectively transitionable between:
a closed position preventing collected urine from exiting the urine hat through the drain port, and
an open position allowing collected urine to exit the urine hat through the drain port.

15. A method of measuring urine output of a patient, comprising:
collecting urine output from the patient within a urine hat of a urine output measuring system;
determining a load value exerted on a platform of the urine output measuring system by the urine hat, the platform coupled with a toilet bowl; and
determining a volume of the collected urine output from the load value.

16. The method according to claim 15, further comprising correlating the volume of collected urine output with a time of day.

17. The method according to claim 16, further comprising transmitting the correlated volume of collected urine output to one or more of a monitor of the urine output measuring system or an external computing device.

18. The method according to claim 16, further comprising transmitting the correlated volume of collected urine output to an electronic medical record system.

19. The method according to claim 15, further comprising calibrating one or more sensors of the urine output measuring system.

20. The method according to claim 15, further comprising:
generating an alert pertaining to operation of the urine output measuring system; and
transmitting the alert to a monitor of the urine output measuring system and/or an external computing device.
